(19) United States
(12) Patent Application Publication
(10) Pub. No.: US 2008/0095705 A1
(43) Pub. Date: Apr. 24, 2008

(54) Methods and Devices for Facile Fabrication of Nanoparticles and Their Applications

(76) Inventors: Jorma Antero Virtanen, Jyvaskyla (FI); Pasi Keinanen, Jyvaskyla (FI); Mikko Tilli, Jyvaskyla (FI)

Correspondence Address: FILDES & OUTLAND, P.C., 20916 MACK AVE., SUITE 2, GROSSE POINTE WOODS, MI, 48236, US

(21) Appl. No.: 11/667,289

(22) PCT Filed: Nov. 8, 2005
(86) PCT No.: PCT/FI05/00476

§ 371 Date: May 8, 2007

(30) Foreign Application Priority Data
Nov. 9, 2004 (FI) 20041435
Dec. 23, 2004 (FI) 20041656
Jan. 31, 2005 (FI) 20050101
Apr. 21, 2005 (FI) 20050406
Apr. 26, 2005 (FI) 20050429

(51) Int. Cl. A61K 49/00; C09K 11/02; A61L 2/28

(52) U.S. Cl. 424/9.1; 422/119; 252/301.35; 977/774

(57) ABSTRACT

The invention provides devices and methods to fabricate nanoparticles by reverse micelle method. The method allows to fabricate a myriad of high quality nanoparticles in a repeatable way. These nanoparticles include multilayered spherical and rod like particles that may have inorganic, organic, polymeric, biological layers. The invention further provides methods to optimize the quality of the nanoparticles.

### CROSS REFERENCE TO RELATED APPLICATIONS

This application claims priority from Finnish Application Serial No. 20041435, filed Nov. 9, 2004, and Finnish Application Serial No. 20041656, filed Dec. 23, 2004, and Finnish Application Serial No. 20050101, filed Jan. 31, 2004, and Finnish Application Serial No. 20050406, filed Apr. 21, 2004, and Finnish Application Serial No. 20050429, filed Apr. 26, 2004.

### BACKGROUND

1. Field of the Invention

This invention provides devices, methods for the fabrication of nanoparticles and applications for these nanoparticles. The fabrication method is based on accurate control of the microfluidics, and the chemical composition of the reaction mixture. This is made possible by a in-line monitoring system and microprocessor control of the various physical and chemical parameters of the system.

2. Prior Art and Overall Description

Nanoparticles (NPs) are widely used in various applications, which include luminescent particles in optoelectronic devices, luminescent, and paramagnetic particles as biological tags, paramagnetic NPs in magnetic imaging, magnetic particles in data storage, metal NPs as catalysts, polymeric NPs as drug carriers, etc. Several methods have been developed for the fabrication of the NPs, such as solvothermal method, flame deposition, electrospray, solvent dispersion, reverse micelle, and sol-gel methods. Solvothermal method is very good for semiconducting NPs. The size, and also shape of the particle can be determined by selecting the reaction parameters properly. The size distribution is very narrow. The drawback is that the reagents are often hazardous, and the reaction conditions are potentially dangerous with these reagents in low boiling solvents and at high temperatures. Flame deposition, and electrospray require specialized equipment, which have fairly limited throughput. Sol-gel method gives NPs in a solid matrix, and its use is limited into the cases, when NPs can be used in that form. Most of the methods are limited to one or two types of NPs. Solvothermal is limited to semiconducting NPs although it gives good quality particles, electrospray, and solvent dispersion are good for polymeric NPs. Flame deposition is applicable to the materials that can tolerate flame without oxidation or decomposition. The only method that is universally applicable to all types of NPs is reverse micelle method. It does not require expensive instrumentation, and is easily scalable to the industrial scale. The problem with reverse micelle method is that it often gives large size distribution.

In the reverse micelle method a detergent is solubilized with a small amount of water into an organic solvent. The detergent should preferably have a small polar headgroup area as compared with the area of the cross-section of the alkyl tail(s). The water will form nanosized droplets inside the detergent shell. The water phase may contain water soluble compounds, such as salts biomolecules, or monomers. These may be solubilized with the detergent at the beginning, or they may be added after the reverse micelles have been fabricated. Also organic solvent may contain some chemicals, such as monomers.

Often a chemical reaction is performed during the fabrication of the NPs. The reagent may be added in water, or some other solvent. With reverse micelles it is possible to perform a reaction between two salts 101 and 102, such as cadmium chloride and sodium sulfide, in organic milieu. The unit reactor is one reverse micelle that limits the size of the particle into nanosize. However, the nanoparticles 103 may react further with starting materials 101 and 102, and the particle size will increase (FIG. 1A-C). The whole sequence of the growth of a nanoparticle is shown schematically in FIG. 1D so that various sizes of nanoparticles 103, 104, and 105 will be formed.

At the end the NPs are coated with some molecules that prevent their decomposition by water, oxygen, and other molecules, and also their sticking with each other.

Microfluidic reactors will solve the problems of the NP fabrication in reverse micelles and other systems (Edel J B, et al., Controlled synthesis of compound semiconductor nanoparticles using microfluidic reactors, Transducers '03 International Conference on Solid-State Sensors, Actuators and Microsystems, Digest of Technical Papers 12^{th}, Boston, Mass., US, Jun. 8-12, 2003, 2, 1730-1733). The particles 103 are immediately removed after the reaction, and they can not react with the starting materials 101 and 102 any more (FIG. 3). This is well known in the art (Nakamura H, et al., Chem. Commun. (2002) 2844, Shestopalov I, et al., Multi-step synthesis of nanoparticles performed on millisecond time scale in a microfluidic droplet-based system, Lab on a Chip 4 (2004) 316-321).

Mixing of components in nano- and microfluidic systems is difficult, because the flow is laminar (FIG. 2). In large containers the mixing is based on convection and turbulence. In microfluidic systems diffusion of the components is the basis of mixing. For that purpose the interfacial area of two components must be maximized so that diffusion between two phases is efficient. This invention provides methods to increase interfacial area so that the microfluidic system itself is simple, and easy to fabricate, and no excessive pressure is required. In addition to increased interfacial area, the mixing can be accentuated by ultrasonic vibration that forces particles to move fast and sometimes to opposite directions even, if they are close to each other. This creates a very strong local mixing effect. The combination of these two methods facilitates the mixing, and the following reactions in microfluidic system so that the throughput, and quality of the products will increase. Although these methods are universal, they will be especially well suited for nanoparticle fabrication.

This invention solves the problems associated with the reverse micelle and other methods in NP fabrication.

### SUMMARY

The present invention provides methods to perform efficient mixing and reactions in nano-, and microfluidic systems. The mixing can be achieved by increasing the interfacial area of two phases by alternating the addition of two or more components into the capillary or mixing chamber. Another method to increase the mixing, and reaction is ultrasonic vibration. These two methods can be used separately or collaboratively.

One embodiment of this invention provides devices for the increase of the interfacial area in nano-, and microfluidic systems by alternating the addition of the components. Controlled addition of the components can be performed with many methods known in the art. These include membrane and piston pumps, stepper motor controlled syringes, electromagnetically controlled bellows, piezo and solenoid driven jets. In order to increase the capacity several nozzles may be used in one mixing chamber.

It is a further object of the present invention to increase the interaction of the reactants by increasing their kinetic energy by ultrasonic vibration. Ultrasonic vibration is most efficiently generated by a piezo crystal that may be outside the fluidic system and by physically connected to it by solid or liquid material, or it may be inside the fluidic system, or it may be integrated with the walls of the fluidic system.

In still another aspect of the present invention, several reagent additions and mixing, and reactions can be performed sequentially in a (micro)fluidic system that has coordinated pumping for these reagents in various joints. This will enable the fabrication of multilayered nanoparticles.

Another purpose of the present invention to provide large arrays of microfluidic continuously operating chips so that industrial scale production is possible.

It is one purpose of the present invention to provide luminescent, paramagnetic, and polymeric nanoparticles, micelles, and liposomes for biological and medical applications.

Still another purpose of the present invention is to provide luminescent, electrically conducting, magnetic, and paramagnetic nanoparticles for optoelectronic applications.

It is an additional object of the present invention to provide continuous fabrication methods and devices for the fabrication of nanoparticles using either alternating reagent addition, ultrasonic mixing, or both.

In one embodiment of the current invention the quality of nanoparticles can be continuously monitored by integrating optical inspection methods, such as UV/Vis, and fluorescence spectroscopy with the microfluidic system of this invention. The inspection system has been connected via a microprocessor to the pumping mechanism so that the reagent addition can be continuously optimized for the best quality.

In still another embodiment of the current invention the inspection is magnetic, so that the magnetic, and paramagnetic properties of the particles can be optimized.

### FIGURE CAPTIONS

FIG. 1. Schematic depiction of bulk fabrication method of nanoparticles. A. At the beginning of the addition the particles are uniform. B. In the middle of addition some particles have grown. C, At the end the product is heterogeneous.

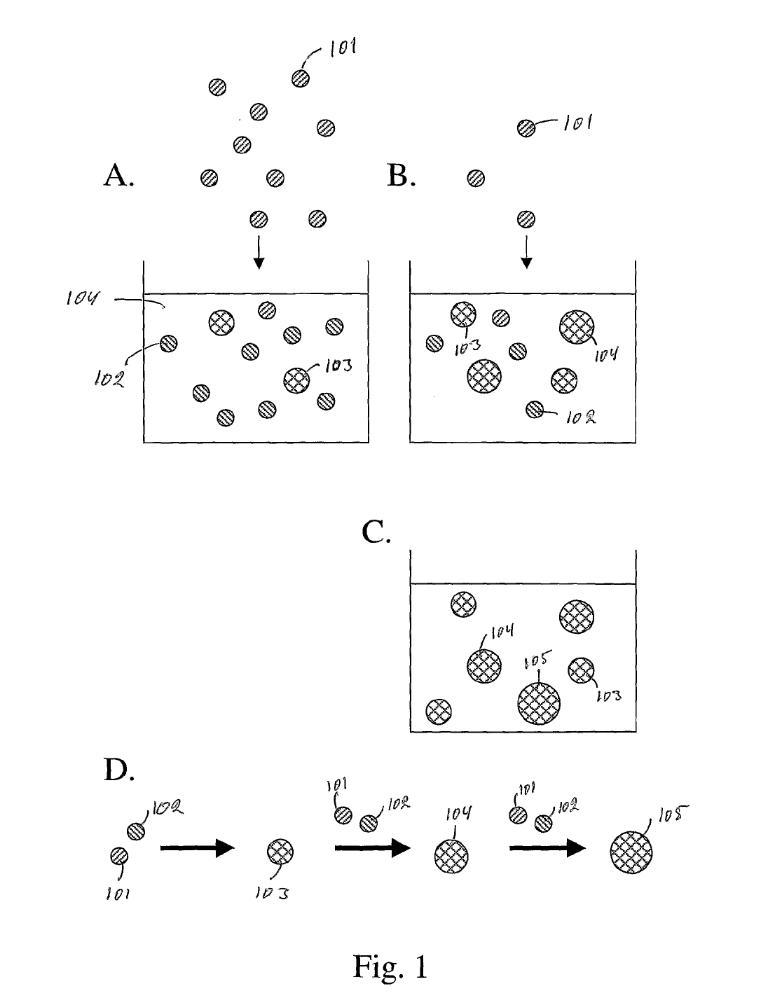

FIG. 2. Schematic depiction of conventional microfluidic addition of two components. Laminar flow keeps both components on their own side, but due to diffusion mixing happens in the central volume.

FIG. 3. Schematic depiction of an ideal stoichiometric reaction near the microfluidic joint.

FIG. 4. Schematic depiction of alternating addition of two components. The zones have paraboloid shape.

FIG. 5. Schematic depiction of hydrodynamic focusing that eliminates some of the drag caused by the walls of the capillary.

FIG. 6. Schematic depiction of conical enlargement of the capillary after the two components have been added by alternatively. The layers get thinner, and mixing is more efficient.

FIG. 7. Schematic depiction of sinusoidal addition of two components under hydrodynamic focusing.

FIG. 8. Schematics of a microfluidic reactor that has three sequential reagent addition joints.

FIG. 9. Schematic depiction of one embodiment of (micro)fluidic reactor that has four reagent addition joints, and corresponding mixing chambers, which are in ultrasonic bath, and one coiled reaction chamber that may be in ultrasonic bath with temperature control. In addition the microfluidic system contains dialysis unit and optical inspection unit that may measure absorbance, luminescence, scattering or some other optical property.

FIG. 10. Schematic depiction of one embodiment of this invention. Reagent and the product collection flasks, micropumps, valves, tubing, and optical detection system are shown.

FIG. 11. Schematic structures of some resorcarenes.

FIG. 12. Janus(a-b-c) molecules that were used for the fabrication of the liposomes.

FIG. 13. Size distribution of the liposomes that were fabricated from the janus molecules.

FIG. 14. Magnetic separation of the affinity bound dual beads, and optical detection of a reporter bead.

FIG. 15. Dual luminescence from a dual bead.

FIG. 16. A. Detection of luminescence from ear lobe. B. Correlation between two emission wavelengths.

### DETAILED DESCRIPTION OF THE INVENTION

Microfluidic System

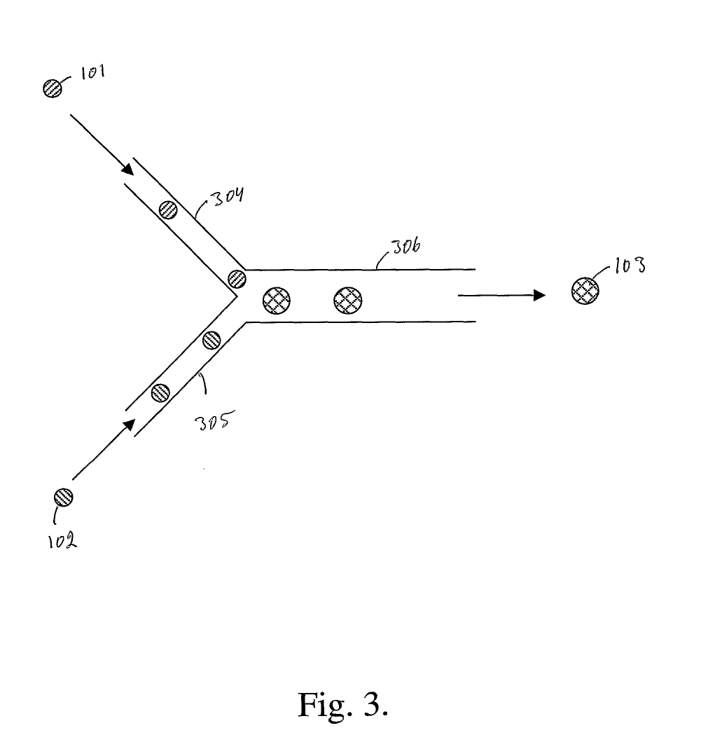

The solution for the size distribution problem, and also for quality heterogeneity problem, is to perform the reactions in a microfluidic, or nanofluidic system, whereby the product 103 is promptly removed, and is not able to react further with starting materials 101 and 102 (FIG. 3). Starting materials 101 and 102 are added through capillaries 304 and 305, and the product 103 is removed through a capillary 306. Microfluidic system as such might not give optimal results. Microfluidic methods for the fabrication of nanoparticles are well known in the art. This invention has several specific embodiments, which will further increase the utility of the microfluidic systems in the NP fabrication.

Controlled addition of the components can be performed with many methods known in the art. These include membrane and piston pumps, electric motor, such as DC or stepper motor controlled syringes, electromagnetically controlled bellows, piezo and solenoid driven jets. In order to increase the capacity several nozzles may be used in one mixing chamber.

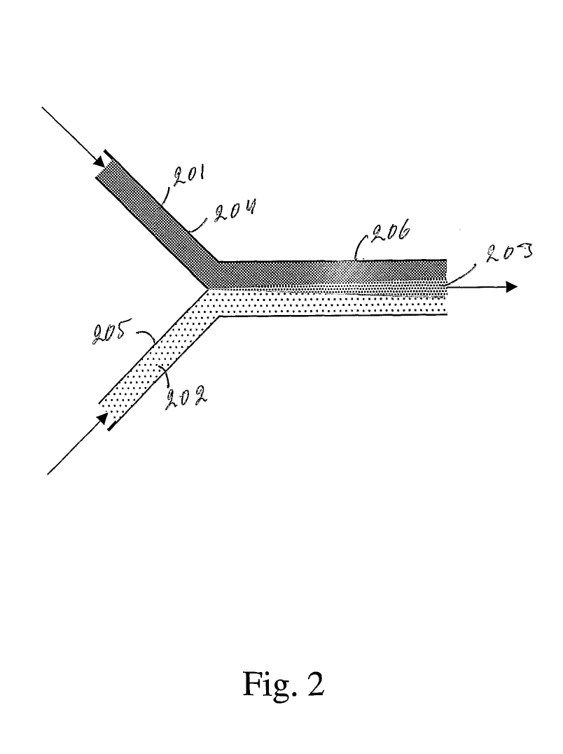

Mixing of reagents must be fast also in microfluidic system. The problem with microfluidics is that the flow is laminar, and there is no turbulent mixing. Ultimately, the mixing is diffusion based (FIG. 2). Two components 201 and 202 are pumped at about the same speed into Y-shaped structure 204, 205, and 206. When they encounter, they will very nicely flow on their own side in the capillary. In the center of the capillary they will gradually mix because of the diffusion. Mixing surface 203 is parallel with the flow direction. However, the product is also in this case continuously exposed to the reactants, and this kind of structure is not much better than a bulk reactor.

Various structures may be used to bring the components so close that they reach with each other in seconds. These structures have a flow resistance that generally is not a problem in small scale fabrication. In large scale fabrication thousands of chips might be run parallel with high flow rate. Then all kinds of mixing structures will create problems.

One way to solve the mixing problem is to use somewhat enlarged mixing chamber, into which the components are added through capillaries. The mixture will be in rotating movement in the mixing chamber that has one exit capillary that is also a reaction capillary. The components will enter into the reaction capillary either as a (nearly) homogeneous mixture, or alternating zones. Further mixing of the components can be achieved by the methods of this invention that will be described in the following.

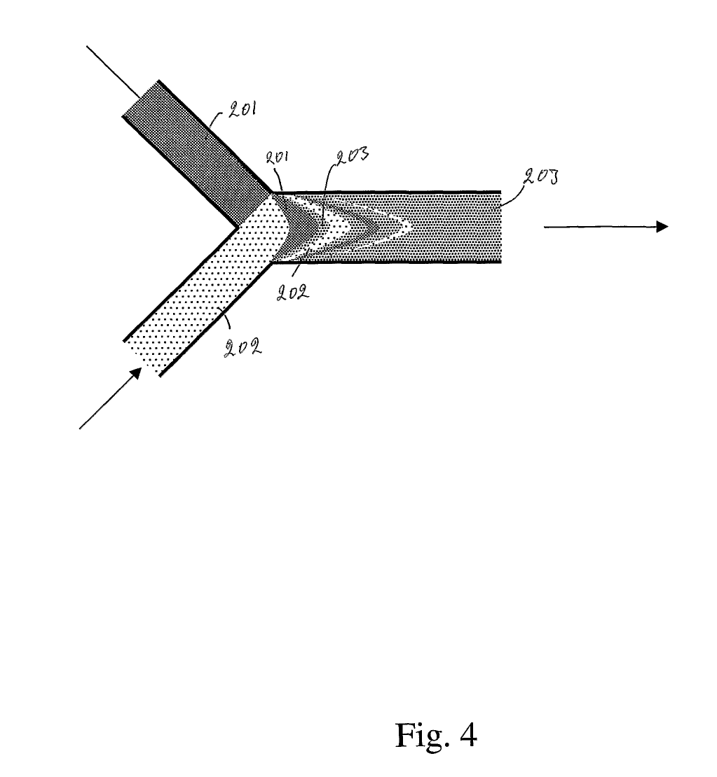

The mixing method of this invention is especially well suited for large scale fabrication. The components 201 and 202 will be pumped alternatively into the mixing capillary (FIG. 4). For instance, if a stepper motors are used for pumping, the first stepper motor will take one step and pump a small amount of the first component 201 into a mixing capillary, and stop. Immediately after that, the second stepper motor will take one step, and pump a small amount of the second component 202 into the mixing capillary and stop, etc. The result is that the mixing chamber will have alternative very thin layers of two reagents 201 and 202. Those layers will mix with each other by diffusion mechanism, and the reagent layers are separated by a reaction mixture layers 203. The mixing is now perpendicular with the flow. The mixing surface area can be much larger than in parallel mixing, and the contact are can be made larger or smaller by adjusting the volume that is pumped in one cycle, and by the thickness of the mixing capillary. The layers are not planar due to friction of the surface, but they are paraboloid shaped. The longitudinal cross-section is a parable (FIG. 4)

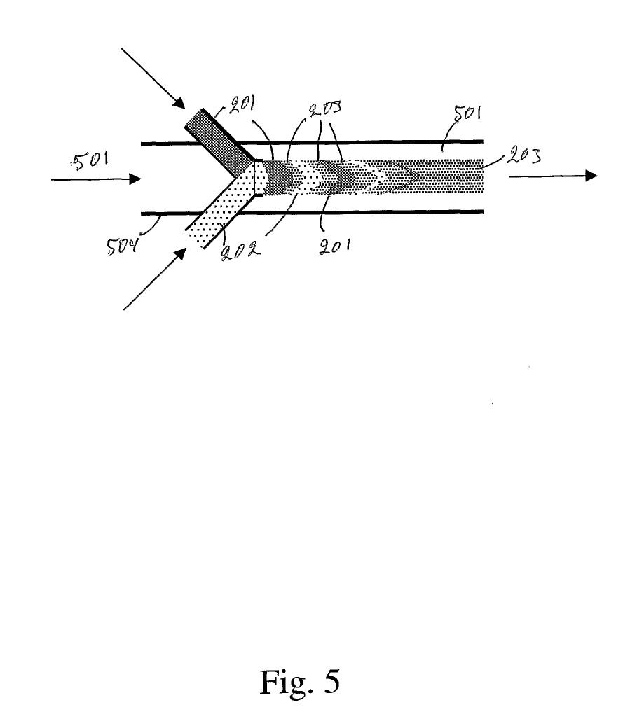

The velocity near the walls of the capillary (or tube) is very low, and the reaction kinetics near the wall and in the center can be different. This will lead also into slightly different kind of product. This problem can be largely avoided by hydrodynamic focusing that is currently used in conjunction of cell counting and well known (FIG. 5). Hydrodynamic focusing can also be implemented with the current invention. In this method a pure solvent 501 is pumped into the perimeter of the capillary or tube 504 parallel with the reagent solutions (FIG. 5). Thus, the reagent flow will not slow down near the wall of the capillary or tube 504. In conjunction of the present invention the volume of the pure solvent is not very large, when compared with the reagent solutions. The goal is to prevent the slowdown of the product near the walls, so that the product 103 can not react with the reagents 101 and 102 significantly.

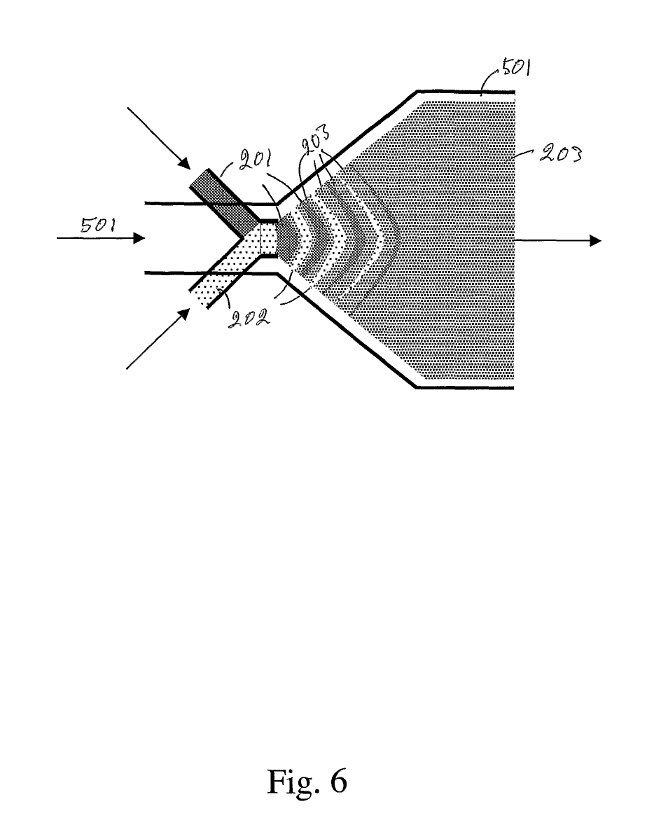

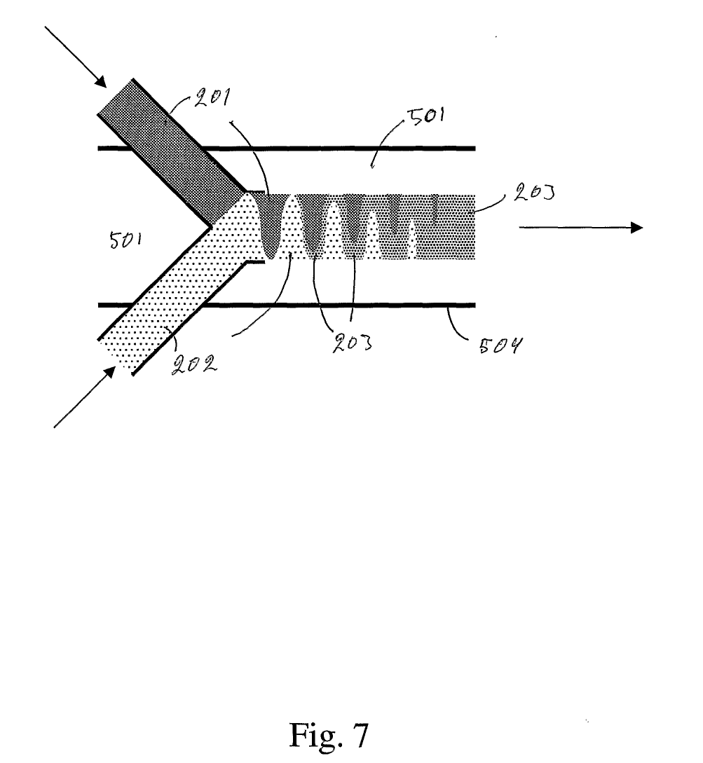

The reagents may be added by several alternative fashion, for example, sinusoidal pumping of the components 201 and 202 is often applicable (FIG. 6). In the example that is depicted in FIG. 7, the sinusoidal addition is combined with the hydrodynamic focusing, in which the reagents 201 and 202 are injected into a solvent 501 that is flowing in a larger capillary 504.

The initial mixing is preferentially done in a fairly small capillary, currently 0.1-5 mm is preferred, but dimensions can be between 10 μm and 10 cm. If the dimension is smaller than 10 μm the perpendicular and parallel mixing give almost equal results. Other aspects of this invention will still improve the product quality. The circular cross-section for the capillary is currently preferred, but the cross-section may be triangle, cut triangle, rectangular, or almost any other shape. The diameter of the capillary may not be constant. On the contrary, in one advantageous embodiment the cross-section of the capillary will increase very soon after the components have been mixed. The increase of the diameter is significant, and can be as much as 100 fold (FIG. 6). The component layers 201 and 202 will get thinner, when the capillary gets wider. The thickness is inversely related to the area of the cross-section. If the diameter is ten fold, the area is hundred fold, and the thickness of each layer is one hundreds of the original thickness, i.e., immediately after entering the mixing capillary. If the thickness of each reagent layer is 0.5 mm originally, they will have a thickness of 5 μm, when the diameter of the mixing capillary (tube) increases ten fold. A complete mixing will happen in seconds.

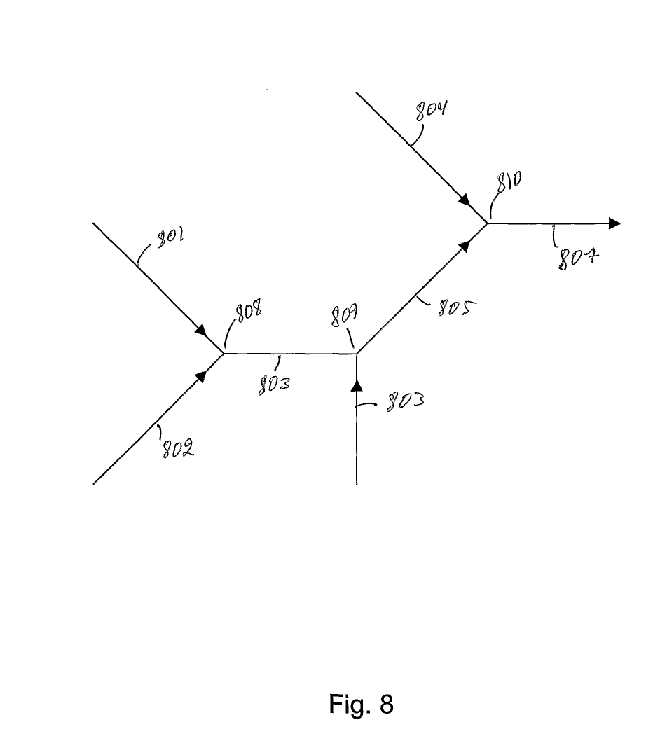

The microfluidic system can contain several reagent addition joints, and mixing and reaction chambers. In FIG. 8 is a highly schematic representation of a system that has three mixing chambers 808, 809 and 810, and four reagent addition capillaries 801, 802, 804, and 806. The reactions happen in capillaries or cavities 803, 805, and 807. One currently preferred application for this kind of microfluidic system is to continuously adjust the concentration of the first reactant by mixing a concentrated and dilute solutions in a mixing chamber 808. The second reactant is added into the mixing chamber 809. A third reactant that may be capping or coating agent is added into the mixing chamber 810.

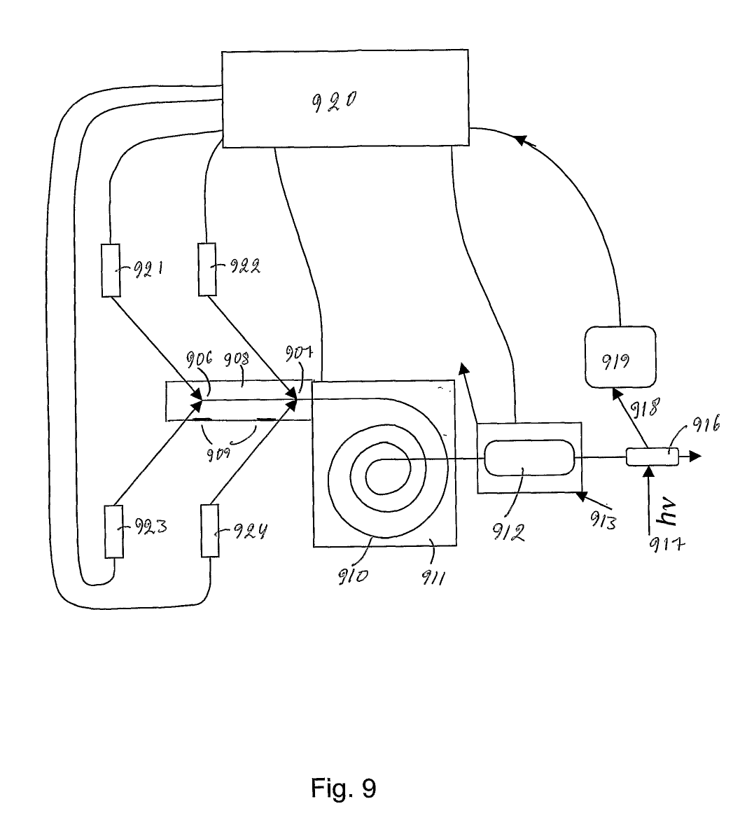

In one embodiment of the present invention the mixing chambers 906 and 907, and reaction capillaries can be vibrated with a piezo crystals 909 (FIG. 9). The frequency range may be between 10 kHz and 500 kHz. Currently 20 kHz-40 kHz is preferred. The ultrasound will increase mixing and reaction rate between the reactants. Vibration source can be outside or inside the capillary (or tube). The piezo crystal may be integrated with the walls of the capillary or container, or via a liquid bath 908 as is depicted in FIG. 9. Vibrating filaments, such as carbon nanotubes, titanium or titanium alloy fibers, may be connected to the piezo crystal to enhance the mixing, if they are inside the microfluidic system.

The temperature of the mixing capillary 910 (or tube) is preferably adjustable between −100° C. and +300° C. When water is one component, the temperature range is between 0° C. and +150° C. Higher temperatures may require closed system that is operated under pressure. The heating can be done by thermal bath 911, resistor, electromagnetic radiation, especially microwave radiation, and sometimes by magnetic induction.

The nanoparticles may be dialyzed immediately after their fabrication (FIG. 9). The product transverses through a dialysis tube 912, and the water, buffer, or some other liquid 913 transverses into the opposite direction outside the dialysis tube. Finally, the quality of the product is measured. In the specific example of FIG. 9 the measurement is done by optical means. The light beam 917 is directed into a flow-through cell 916, and the light 918 that is coming out from the cell into a certain direction is measured. That light may be the light that is not absorbed, it may be scattered, fluorescent, or luminescent light. The information is collected by a microprocessor 920. The microprocessor 920 will control every operation of the device, including the reagent addition pumps 921-924.

The most advanced embodiment allows to control virtually all relevant physical and chemical parameters, including continuous measurement of chemicals, temperature, pressure, vibration, electric and magnetic field, light intensity, wavelength, and polarization.

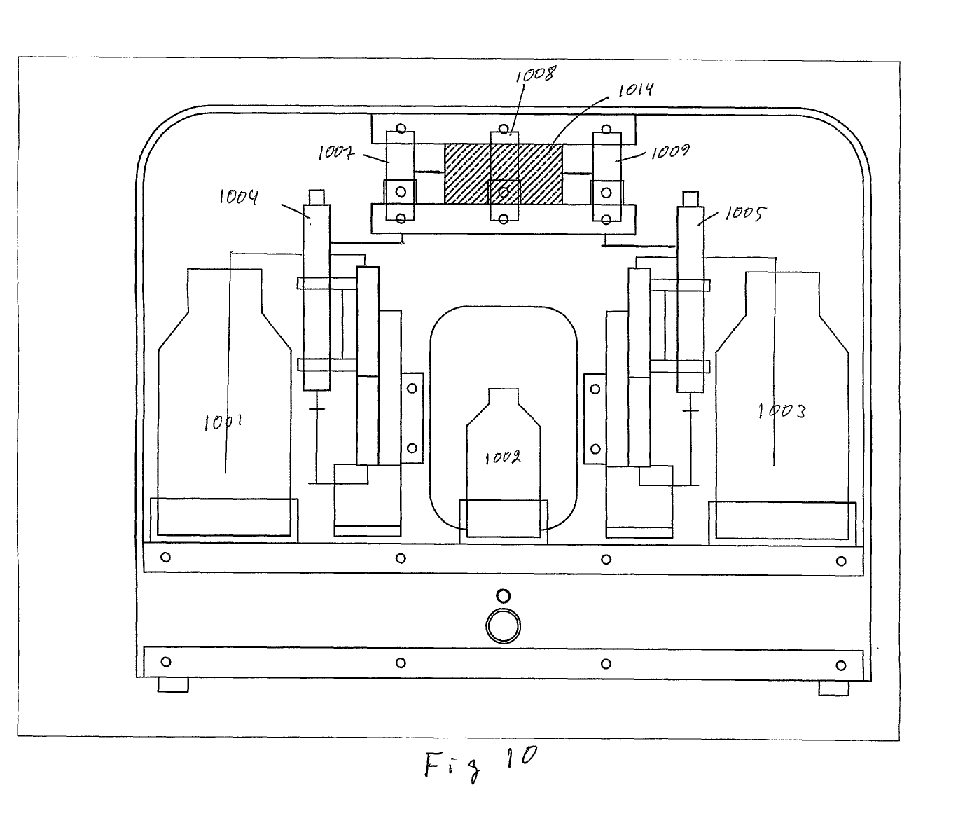

A drawing of one currently used embodiment is shown in FIG. 10. The reagent flasks 1001, 1002, 1003, syringe pumps 1004, 1005, valves 1007, 1008, 1009, tubing 1011, 1012, 1013, and optical measurement unit 1014 are shown. Microprocessor controls all operations and collets the data. Microprocessor is not shown in FIG. 10.

Process Optimization and Control

The formation of nanoparticles is advantageously followed in real time by on-line spectrophotometer, spectrofluorometer, light scattering, magnetometer, electrical conductivity, or dipole moment measurement, or any combination of these. The information will be processed by a microprocessor that can change the injection velocity of the reactants. During optimization the changes are performed systematically, until a steady-state is found.

The desired excitation and emission wavelengths are generally known before optimization. In that case the fixed values of these parameters can be set in the instrument. The reactant vessels are loaded with appropriate reagents. The concentrations are set to the high end, because the solutions can be diluted during the injection of the reactants by adding solvent. The computer is programmed so that all desired combinations of reactants are tried. These combinations and related absorption and emission properties are saved into the memory. The absorption and emission spectrum can also be measured to optimize the peak widths.

The optical unit can also measure absorbance, turbidity, or light scattering. Turbidity and light scattering depend strongly on the particle size, Especially, when the total amount of material is known, these optical properties are sufficient for the size measurement. To obtain more accuracy these optical properties can be calibrated for each type of particle by transmission electron microscope or atomic force microscope imaging.

Similarly, magnetic properties can be optimized by measuring the magnetic properties of the particles. For that purpose a capillary is surrounded by a coil that is connected to a current or voltage measurement unit. Two sequential coils that are coiled in opposite direction give very accurate result. It is advantageous, if the capillary is thin so that particles move very fast inside the coil, and also the coil is very close to the particles. If particles are paramagnetic, a permanent magnet is needed to magnetize the particles, while they are being measured.

In the very important embodiment of this invention the measurement result is recorded, and a microprocessor will control reagent addition, and the relevant physical parameters of the reaction, so that the quality of the nanoparticles stays in the preset limits. These limits include the absorption and emission wavelength, light scattering, and magnetic properties. For instance, in the fabrication of cadmium sulfide nanoparticles the desired emission wavelength maximum can be 520 nm, and the allowed range 516-524 nm.

The device can also search reaction conditions that give the desired properties for the particles. Various search algorithms may be used for this purpose. A systematic search is one possibility. In this case the device will automatically study all combinations according a preset table. Another possibility is to change one variable into one direction, and then to another, and choose the better of these two. A larger change is performed into the favorable direction. Now small changes are done into both directions. If the better direction is the same as the first time, another large change is done into the same direction, etc. until the opposite direction gives the better result. Then this variable is changed back halfway of the last large step. Optimization can be continued to a desired accuracy, but the highest accuracy is not necessary, before all variables have been adjusted. After the first variable is optimized roughly, the second variable will be optimized similarly, etc. Once all parameters are nearly optimized, the whole optimization cycle is repeated more accurately.

Reverse Micelles

The reverse micelles (RMs) are spherical, rod, or disc like depending of the detergent. Most detergents, such as sodium bis(2-ethylhexyl)sulfosuccinate (AOT), give spherical RMs, while hexadecyl trimethyl ammonium bromide and detergent mixtures that contain phospholipids like 1,2-dilauroyl phosphatidyl ethanol amine give elongated, or rod reverse micelles. These features may be further amplified during the growth of the nanoparticles. The detergent may be the same for various starting materials, or it may be different. For example, AOT may be used for cadmium nitrate solution, while, hexadecyl trimethyl ammonium bromide may be used for sodium sulfide, i.e., anionic detergent for a cation, and cationic detergent for an anion. Alternatively anionic detergent may be used for an anion, and cationic detergent for a cation.

Organic solvent can be almost any aliphatic or aromatic liquid hydrocarbon, such as hexane, cyclohexane, iso-octane, toluene, or halogenated hydrocarbon, such as dichloromethane. Also ethers, such as dibutyl ether are applicable. The main requirement is that the detergent or water must not be too soluble into the solvent.

The most basic method is to form insoluble salt from two soluble components. Examples are plentiful, and include formation of silver halogenides from silver nitrate and alkali halogenides, copper, lead, mercury, iron, nickel, cobalt, zinc, cadmium sulfides from corresponding nitrates or chlorides and sodium sulfide, barium sulfate from barium chloride and sodium sulfate, etc. In these cases two reverse micelle solutions are prepared and these are mixed according to this invention. The salts can be added as water solutions after the fabrication of the RMs, or at least one ion may for a salt with anionic or cationic detergent, for instance cadmium-AOT₂.

The size of the RMs can be estimated from the molar ratio water to detergent W₀. The radius is approximately for spherical RMs

$$R = \frac{{W}_{0}}{11 a}$$

where a is the area of the detergent exposed inside the RM. In the following examples we suppose that a=0.5 nm². Also CdS is used as a specific example in the calculations. The water phase may contain 1 M or 0.2 M cadmium nitrate, for example. The radius of the CdS nanoparticle that could be formed from the cadmium nitrate that is inside one RM is R/3.2 for 1 M solution, and R/5.5 for 0.2 M solution. Thus, in order to make a CdS nanoparticle that has a radius of 2 nm from the contents of just one RM the radius R should be 11 nm, if the cadmium nitrate concentration is 0.2 M. This will require that W₀=60. This is quite high and impractical value for W₀. The W₀ that is currently often used to obtain the radius 2 nm for the CdS particles is about 4. These RMs have a radius of 0.73 nanometers, if they are spherical, and contain 0.2 cadmium ions per RM on the average, and similarly for sulfide ions. Thus, currently the contents of about 3000 must be combined to get the cadmium for just one nanoparticle that has a radius of 2 nm.

One way of protecting of CdS, and similar nanoparticles, is to cap them with alkane thiols. The molar ratio of the capping molecule to the cadmium S is related to the ratio of the particle r by the equation

$$S = \frac{0.74}{r} .$$

The capping molecule can be added after the particles are ready, or already during the growth. The numerical constant (0.74) depends inversely on the area of the capping reagent on the surface. Also in the derivation of this equation it is supposed that the binding between the nanoparticle and capping molecules is quantitative. However, the binding constant is often relatively small, and the equilibrium between bound and free capping molecules must be taken into account.

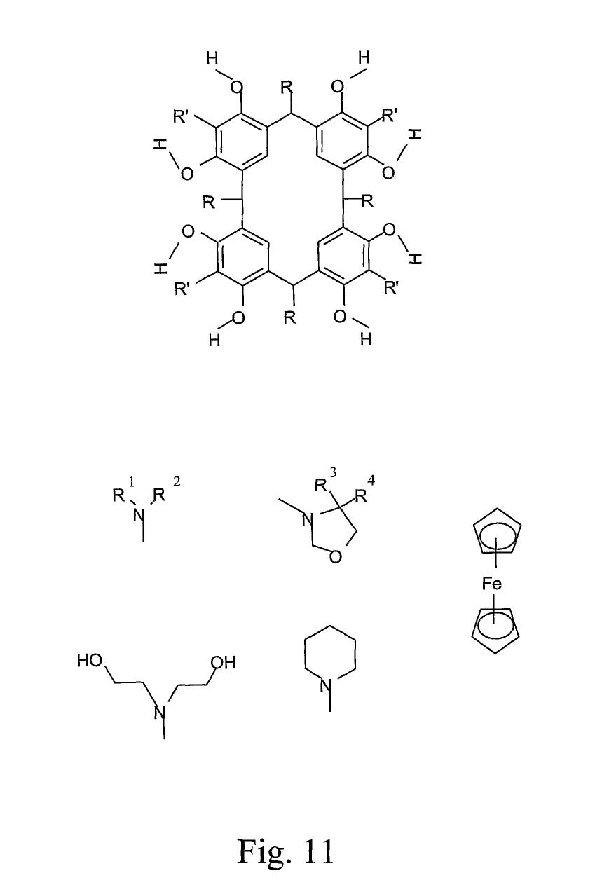

Addition of the capping compounds at the beginning, such as alkanethiols, merkapto ethylamine, or even thiol containing or thiolated biomolecules will stabilize nanoparticles already during their growth. We have found that macrocyclic compounds that contain amino, thiol, phenolic, or acidic functionalities are excellent capping molecules. Resorcarenes are examples of this class of compounds (FIG. 11). The substituents can include electronically or optically active moieties, such as ferrocene.

At least two different mechanism are important for nanoparticle formation in reverse micelles. First, two or more micelles can fuse, at least temporarily, and exchange their contents. Second, one or more reagents can be solubilized in an organic phase, or travel through an organic phase with the help of a phase transfer agent that can be a detergent. The relative importance of these mechanisms can be adjusted within wide range by the choice of the detergent(s), codetergent, solvent, concentrations, temperature, vibration, and electromagnetic field.

The reverse micelle method is widely applicable to the fabrication of the NPs. The particle size distribution is often too large, and the quality of the particles is not always satisfactory. One of the most important reason for this is that the reverse micelles that contain reagents will combine with the reverse micelles that contain the product several times. Actually, the combination may happen tens, hundreds, or even thousands of times. If the reactant particles donate their contents only partially with each encounter the increase can be slow. Also the formation of multicrystalline nanoparticle is possible. In any case, the size distribution is significant, continuous, and will actually prevent the use of these NPs in many applications.

Solvothermal and Colloidal Method

Solvothermal and colloidal methods are also applicable in the microfluidic system of this invention. The high temperatures that are used in solvothermal and colloidal methods require that the fluidic system is in a pressurized chamber, or alternatively, high boiling solvents and reagents are used. One or more of the reagents are often solid and sparingly soluble. In these cases it is preferred that the solid is in a cavity 1020 (FIG. 10), and the solvent is heated just before it enters that cavity. This method is used for all sparingly soluble reagents. The hot solutions are mixed after the solubilization step. In some cases one or more component is very poorly soluble, and the formation of nanoparticles is essentially heterogeneous process. In these cases all necessary reagents may be mixed in the same cavity and hot solvent is pumped through this cavity, or alternatively several cavities that have different temperature control may be preferred.

Examples of these methods are the formation of indium phosphide nanoparticles from indium trichloride and trisodium phosphide at 180° C., when tri(ethyleneglycol) dimethyl ether (b.p. 216° C.) is used as a solvent. All reagents should be mixed in the fabrication of indium arsenide from indium trichloride, arsenium trichloride and zinc at 180° C. in tetralin. Colloidal method is closely related to solvothermal method. The main difference is the use of a detergent, such as tetradecyl or octadecyl phosphonic acid. The detergent allows easier solubilization of some starting materials, and the nanoparticles. Also the size control is easier than in solvothermal method.

Another important example is the fabrication of cadmium selenide nanoparticles. Dimethyl cadmium is a soluble source for cadmium, but it is extremely poisonous, and cadmium oxide is currently preferred. Cadmium oxide and selenium are placed in separate heatable containers in Y-tube system so that different solvents and temperatures can be used for each reactant. The solutions merge, and the reaction happens. Suitable solvents are trioctylphosphine and octadecyl phosphonic acid. The temperature is close to 300° C.

One currently preferred method of this invention is closely related to solvothermal and colloidal method, except that no reaction is performed. Instead, a premade material, for example cadmium selenide, is slowly dissolved into a hot solvent in the presence of a detergent. The flowing solution will be gradually cooled down and the nanoparticles will be deposited. Solvents and detergents can be the same or analogous to those that are used in the conjunction of the solvothermal and colloidal methods.

Chemical Methods

Almost any kind of nanoparticles can be fabricated with this method. Luminescent quantum dots are one very important, because their properties are critically dependent on the dimensions, structure, and coating. Other types of particles include, metal particles such as gold, nickel, cobalt, and molybdenum, paramagnetic particles, polymeric particles, and particles that contain biomolecules such as enzymes. Most of these particles are well known in the art.

Traditional inorganic solution deposition methods are applicable almost for all sparingly soluble compounds. For example silver chloride can be prepared by mixing silver nitrate and sodium chloride solutions. Similarly, numerous other metal halogenides, sulfides, oxides, phosphates, sulfates, carbonates, and other compounds can be fabricated. In the fabrication of nanoparticles the solutions should be dilute enough so that crystals do no grow large. The present microfluidic method has again significant merits. The nanocrystals that are produced are immediately removed so that the new reagents can not deposit on their surface.

Nucleation rate should be as high as possible, because the bigger the number of the crystals, the smaller they are. Nucleation rate can be increased by increasing the concentration of one reagent. Also some solvents like ethanol might favor higher nucleation rate than water, because ions are more weakly solvated. Ultrasonic vibration promotes nucleation, because the local movement of the ions is increased.

The growth of the nanocrystals can be slowed down by capping reagents. Oligomeric or polymeric capping reagents, such as polyacrylic acid, polyacrylonitrile, polyethylene glycol and polyallyl amine are currently favored, because they bind larger particles well, but do not have high affinity for individual cations or anions.

We have found that macrocycles are very good capping reagents. For example, many resorcarenes (FIG. 11) are adsorbed effectively on the surface of many nanoparticles. Especially, resorcarenes that have free phenolic groups and/or amino groups are currently favored.

It is not mandatory that all starting materials are salts. For example, the sulfide ion may be generated in situ from thiourea, dimethylsulfoxide, thiocarbonyl diimidazole, or a similar chemical. Metal ions might have ligands or carriers such as EDTA, triphenyl phosphine, bipyridine, and crown ethers.

Another method resembles sol-gel method in regard to starting materials of the nanoparticles. However, the continuous gel part and the associated nanocavities are missing, and the reverse micelles substitute those cavities. At least one of the reagents is first in the organic phase, and when it gets into contact with water the hydrolysis product will deposit either alone or with some other reactant as a nanoparticle. Metal alcoholates are hydrolyzed in water phase into metal hydroxides, which often transform into corresponding oxides either spontaneously, or after heating. In order to neutralize the hydroxide ions, the water may contain acid, such as hydrochloric, hydrobromic, nitric, perchloric, sulfuric, formic, trifluoroacetic, or an easily hydrolysable ester like methyl formate. If the water phase contains anions, such as halogenides, sulfide, selenide, telluride, carbonate, sulfate, phosphate, arsenide, and oxalate a corresponding salt is formed. The organic phase may contain several different alcoholates, and tetramethoxy or tetraethoxy silicate. Either composite nanoparticle, or metal silicate nanoparticles are formed in this case. Typical metal alcoholates or related compounds include barium(II)isopropoxide, zinc(II)methoxy ethoxide, aluminum(III)isopropoxide, iron(III)isopropoxide, nickel(II)methoxy-ethoxide, holmium(III)iso-propoxide, yttrium(III)iso-propoxide, europium(III)-D-3-trifluoro-acetylcamphorate, tin(IV)iso-propoxide, titanium(IV)isopropoxide, titanium(IV)iso-butoxide, tungsten(V)ethoxide, tungsten(VI)isopropoxide, bismuth(III)methoxy-2-methyl-2-propoxide, hafnium(IV)tert-butoxide, hafnium tri-isopropoxy tetramethyheptane-dionate, zirconium(IV)tert-butoxide, niobium(V)isopropoxide, vanadium(V)tri-isopropoxide oxide, aluminium cobalt isopropoxide, lead(II)titanium(IV)isopropoxide, lead zirconium ethylhexano-isopropoxide, and SrTa₂(OEt)₁₀(dmae)₂. Almost any ceramic nanoparticle can be prepared using alcoholates. Examples are lead zirconium titanate, and strontium bismuth thallium oxide nanoparticles, which are piezo electric materials, and yttrium barium copper oxide that is a high temperature superconducting material.

Silylamides are another class of soluble metal compounds that are hydrolyzed by water. These include tris[N,N-bis(trimethylsilyl)amide]-cerium(III), and analogous compounds of erbium(III), europium(III), gadolinium(III), holmium(III), lanthanum(III), lutenium(III), neodium(III), praseodymium(III), samarium(III), scandium(III), terbium(III), thulium(III), ytterbium(III), and yttrium(III). These metal cations are lanthanides, and form fluorescent nanoparticles. Some other metals can be coprecipitated in order to change absorption or emission properties, or simple to reduce the cost.

Many soluble metal ions, or nanoparticles containing reverse micelles, can be reduced into metallic nanoparticles in the microfluidic system of this invention. Reducing agents can be sodiumborohydride, sodium cyanoborohydride, hydrazine, citric acid, ascorbic acid, hydroquinone, and many other ones known in the art. Oxidized metal, and reducing agent containing reverse micelles are mixed as previously described. For example, silver, gold, nickel, and cobalt nanoparticles can be fabricated.

Several metallic nanoparticles may be fabricated from metallo-organic or metal carbonyl compounds by thermal, photochemical, or ultrasonic decomposition. Metallo-organics, and carbonyls include dibenzene chromium, di(ethylbenzene) molybdenium, iron pentacarbonyl, chromium hexacarbonyl, and tungsten hexacarbonyl. Ultrasonic decomposition is currently preferred. This method is based on the decomposition of a single chemical compound. Microfluidic system provides again significant advantages over traditional reactors, because the newly formed nanoparticles are continuously removed, and their growth will stop once they are not subject to ultrasonic vibration. The solution may contain suitable coating reagents that prevents the metal nanoparticles from aggregating. The coating reagents may be, for example, amines, carboxylates, polyethers, polyunsaturated hydrocarbons.

These chemistries allow the fabrication of multilayer nanoparticles. For example, cadmium sulfide particles can be coated immediately with zinc sulfide layer, which can be coated with niobiumoxide layer. Niobium oxide is chosen, because it is sparingly soluble in water. Niobium oxide can be coated with peptide or protein layer. Suitable peptide is poly-L-lysine that is preferable grafted with polyethylene glycol. Also lys-lys-lys-gly-asn-asn-ser type peptides, which have the binding part (lys), joint (gly), and hydrophilic part (asn, ser) are currently preferred. Metal oxide, or sulfide layer can be directly coated with a protein like avidin. Certain mutants, or avidin like proteins, which have cysteine moieties on the surface (Hytonen V, et al. J. Biol. Chem. 279 (2004) 9337, and Reznik G. O., et al., Bioconjugate Chem. 12 (2001) 1000), will bind gold nanoparticles, and also with sulfide, and selenide containing nanoparticles directly. These avidin analogs will also bind onto the gold nanoparticles. All these various layers can be formed in a microfluidic system of this invention. Moreover, all layers can be formed in a continuous process so that components are added sequentially in a long microfluidic system that has one specific joint for each reaction, and possibly for each reagent. Alternatively, the nanoparticles can be recycled through a one joint reactor to form a new layer with new reagents.

Multilayer nanoparticles can have interesting optical properties, such as fluorescence energy transfer, or simultaneous optical filtering and amplification. These particles can be spherical or rod like. For example, a spherical particle can have CdSe core that is surrounded by ZnS layer that is surrounded by a CdSe layer that is surrounded by ZnS layer etc. The thickness of the CdSe layers can decrease monotonically, so that the outmost layer is thinnest. The outer layer has the shortest wavelength absorption edge, and photoluminescence wavelength. Accordingly, the excitation energy will transfer to the inner layer, and finally to the core. This kind of particle will absorb effectively across the spectrum below the core particle absorption, and still have very sharp emission. The first excitation can also be electronic.

Rod like particles can have several layers, too. For instance, every other layer can be optically transparent spacer layer, and the layers between are either filtering, or amplifying layers. The topmost layer should be filtering layers, so that the light has been largely filtered, before it enters into the amplifying layers. There should still be filtering layers between amplifying layers. Filtering layers in the visible area should be nonluminescent, such as salts or oxides of iron, nickel, cobalt, copper, mercury, and other colored nanolayers. Cadmium sulfide, and selenide can be used, if they contain quenching impurities, or neighboring layer is not inert spacer, but a quenching layer. Amplifying layers can be, for example, europium, or rubidium oxide nanolayers that are excited by light or electric potential. When a light that is able to pass through the filtering layers, encounters an amplifying layer, it will cause the excited atoms or ions to relax to the ground state. The emitted light will have the same direction and phase than the light that entered the amplifying layer.

Liposomes and Polymeric Nanoparticles

One well known method for the fabrication of liposomes is ethanol injection. In this process a phospholipids or analogous compound is dissolved into ethanol, and this solution is injected very fast into water or buffer under vigorous stirring. This method is applicable for the device of this invention. The newly formed liposomes are immediately removed from the mixing area, and their size will not increase, when fresh lipid solution is injected. Ethanol or water phase may contain additional compounds, such as drugs, reagents, or enzymes. Ethanol may later be removed by evaporation or dialysis. Dialysis may happen immediately, if part of the tubing is semipermeable. Instead of ethanol some other water soluble solvents can be used. These include methanol, propanol, acetone, dimethyl formamide, and tetrahydrofurane.

Liposomes may be serve as nanoreactors for the fabrication of other nanoparticles. For instance, cadmium salt solution may be trapped inside the liposomes. Cadmium will leak out very slowly. The cadmium may be removed from the outside by ion exchange or dialysis. It can also be bound with strong chelators, such as EDTA. Sulfide may be introduced outside of the liposomes as a component of a salt, such as sodium sulfide, and carried into the liposomes by charge transfer catalyst such as trimethyl octadecyl ammonium bromide. Situation may be reversed, so that sulfide is inside the liposomes, and cadmium is carried inside by crown ethers or analogous compounds. Sulfide may also be generated in situ from compounds that slowly release the sulfide ion. If the cadmium or sulfide concentration is 40 mM, the radius of the nanoparticles is R/9.5, where R is the radius of the liposomes. With 8 mM solution the radius will be about R/16.2. For example, if the radius of the liposomes is 32 nm, the radius of the nanoparticles is approximately 1 nm.

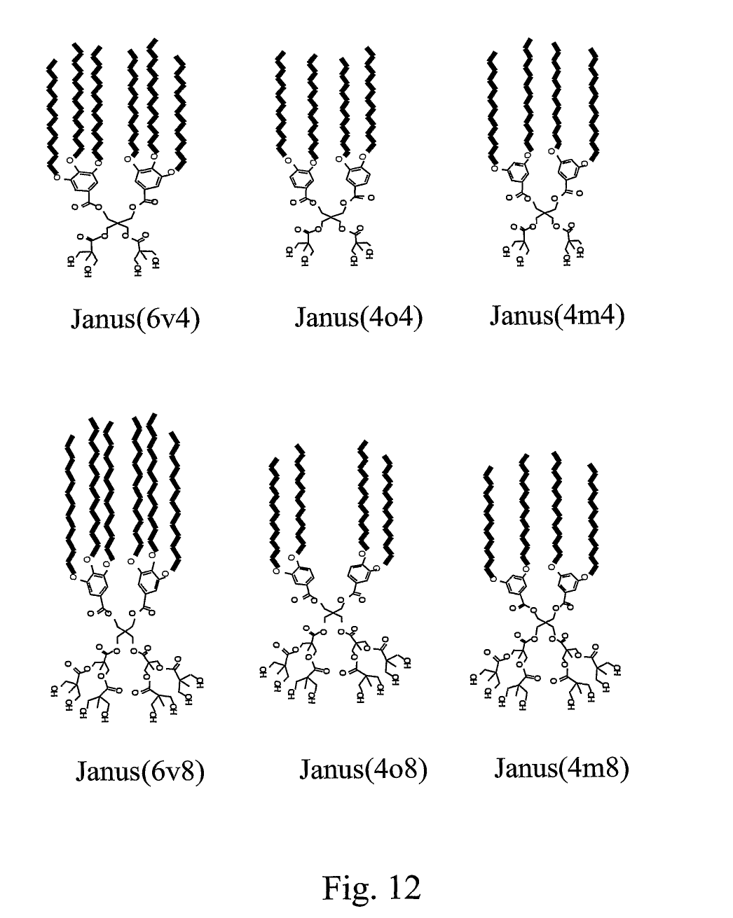

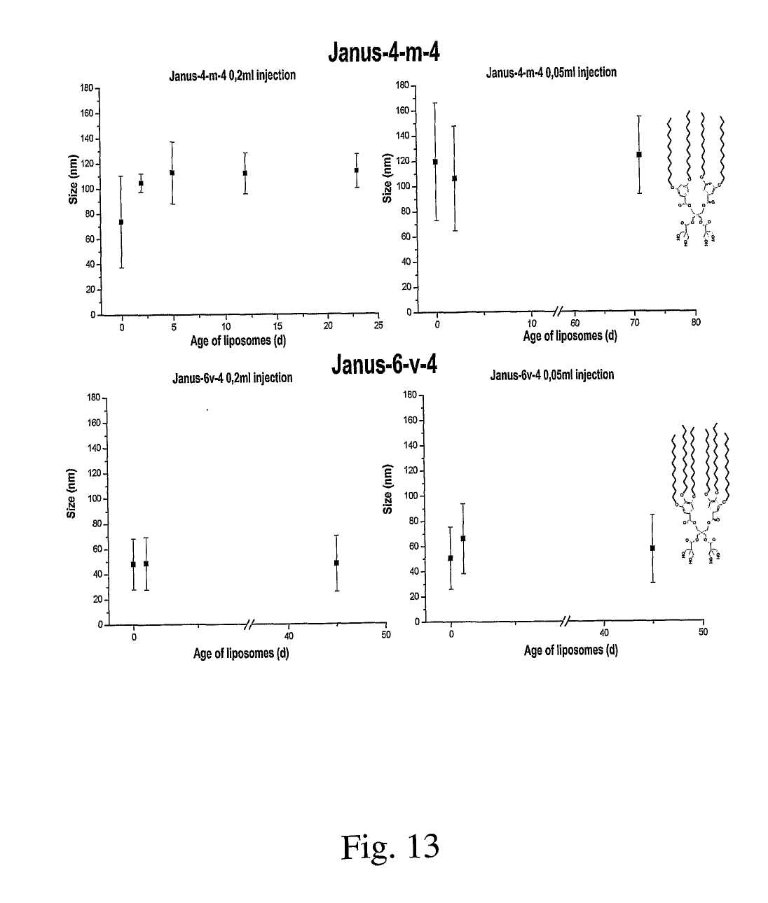

Liposomes can be fabricated from natural or synthetic phospholipids, such as dipalmitoyl-, or palmitoyloleoyl phosphatidylcholine. Often artificial amphiphilic compounds can be better be tailored for a specific application. Stability is major factor in practical applications. Four and six alkyl tail molecules, which have hydroxyl functionalized dendritic polar head group were found to form very stable liposomes. These molecules are named as janus(a-b-c) molecules, where a gives the number of alkyl tails, b indicated the relative location of the alkyl tails in the “neck” area, and c gives the number of the hydroxyl groups (FIG. 12). The liposomes were stable several months (FIG. 13, Example 3). Their size distribution was narrow. These factors make them very good nanoreactors, and drug carriers.

Polymeric nanoparticles may be prepared analogously to liposomes (Brannon-Peppas L, Recent advances on the use of biodegradable microparticles and nanoparticles in controlled drug delivery, Int. J. Phamaceutics 116 (1995) 1-9, Bodmeier R. et. Al. J. Microencapsulation 8 (1991) 161-170). Polymer is dissolved into a water soluble solvent, or in solvent mixture that is mostly water soluble, and that solution is injected into water. Solvents include methanol, ethanol, iso-propanol, butanol, pentanol, acetone, methyl ethyl ketone, ethyl acetate. Again drugs or some other compounds may be in either phase. Biocompatible NPs are preferably prepared from biodegradable polymers, such as polyglycolic acid, polylactic acid, hyalyronic acid polyaminoacids, proteins, and glycoproteins. Detergent may be present during the mixing, or it may be injected later. Detergents will solubilize the nanoparticles and prevent them from sticking. Suitable detergents are, for instance, octyl glucoside, tween, polyethylene glycol esters, triton, sodium dodecylsulfate, and trimethyl ammonium bromide. The choice of the detergent depends on the end use. Fast removal of the polymeric NPs from the mixing area prevents their further growth, and their size distribution is easily controllable. Polymer may be amphiphilic. For example polylactic acid may be chemically bound with polyethylene glycol by an ester bond. The mixture of a polymer and amphiphilic polymer will be used in most applications. The ratio of these components is a major factor determining the size of the nanoparticles.

Polymeric nanoparticles may be fabricated by dispersing monomers into a suitable solvent that may be water, alcohol, ester, ether, hydrocarbon, halogenated hydrocarbon, or ionic solvent. Polymerization may be initiated by nucleophiles, such as amines, electrophiles, such as proton, radicals that are formed by heat or electromagnetic radiation, including UV-radiation, from peroxides or other well known polymerization initiators.

Most drugs have relatively high affinity for polymers, and will be spontaneously adsorbed or adsorbed. By injecting polymer and/or drug several times from consecutive ports, when the solution is propagating in the microfluidic system, layered structures with different drug concentrations and polymers can be fabricated.

NPs of conducting polymers may be prepared similarly from conducting polymers. The problem is the poor solubility of conducting polymers in several solvents. Conducting polymer may be mixed with an additive that may be another polymer, for example, polyglycolic acid, or polyethylene glycol. Another possibility is to fabricate NPs or nanodroplets from the starting compounds, for example, aniline or thiophene, and polymerize them similarly as bulk polymerization is performed. For example, polyaniline NPs may be prepared in reverse micelles or liposomes. In one currently preferred protocol liposomes will be first loaded with 0.25 M ammonium peroxydisulfate. Aniline will be added in ethanol, so that its concentration is 0.1 M. Aniline will penetrate into the liposomes. After half an hour hydrochloric acid is added in the solution to turn aniline into hydrochloride. Lowering the temperature to about 0° C. will increase the conductivity of the polyaniline NPs. Decreasing the pH to about zero will further increase the conductivity. Metallic nanoparticles, such as nickel, silver, and gold NPs, may be coated with conducting monomer or polymer. This kind of coating is advantageously performed with inverse micelle method. Metallic nanoparticles can be first fabricated in inverse micelles by reduction of metal salt with hydrazine, sodiumborohydride, or some other well known reducing agent. For the polyaniline coating aniline hydrochloride, and ammonium peroxydisulfate containing reverse micelles will be subsequently added. Reverse micelles may also contain conductive detergents, such as tetrathiophene carboxylic acid. These NPs may be advantageously be used in printed electronics on flexible substrates, because the conducting polymer will connect the metal particles even, when the substrate is being bent.

Polymer may be deposited onto the surface of some other nanoparticles, such as metal, or semiconducting nanoparticles, or vice versa, metals or semiconducting materials may be deposited on the surface of polymeric nanoparticles. Thus, core-shell particles are created. Also premade metal or semiconducting nanoparticles may be bound onto the polymeric nanoparticles. This aggregate may be further be coated with polymeric, metallic, or semiconducting material.

Postreatment of the Nanoparticles

In many applications the nanoparticles are used as a dry powder, or a water solution. The dry powder is easiest to obtain by evaporating the solvent including the water. It may be advisable to add isopropanol or some other water soluble organic solvent so that the evaporation of the water is smooth. If the nanoparticles are wanted in the water solution, an alcohol-water mixture can be added so that the organic, and water phase will be separated. The water phase may be dialyzed to remove extra salts, and other water soluble reactants, and reaction products.

The water soluble nanoparticles may be further fractionated by gel chromatography that separates the particles according to their size (recommended column is Amersham Superose 6 10/300 GL). Although the method of this invention gives very narrow size distribution, the fractionation will still improve somewhat the optical, and electronical properties of these nanoparticles.

Starting materials, and several impurities can be removed by dialysis. This applies especially to nanoparticles that are in water or in a solvent mixture that is mostly water. The device can have a dialysis tube that is in a tubular bath that has a countercurrent flow of a corresponding pure solvent.

Some nanoparticles, especially ceramic nanoparticles, are often annealed before use. For example, europium oxide containing nanoparticles are preferably annealed at about 700° C., in order to get the optimal luminescence.

The device of this invention allows the fabrication of multilayered nanoparticles. For example, the quantum dots, such as cadmium selenide, may be coated with an inorganic shell, such as zinc sulfide. The inorganic shell may be further be coated with an organic layer, such as 6-mercapto-hexanoic acid. Finally, biological molecules, such as amino-oligonucleotides, may be conjugated with the organic layer. These all steps can be performed without removing the nanoparticles from the microfluidic system. Dialysis is recommended between steps.

Applications

Nanoparticles have numerous applications, and only some can be mentioned. Lanthanides are relatively nontoxic, for example europium oxide has LD₅₀ of 3 g per kg. Thus, europium oxide nanoparticles can be used for monitoring flow of ground or waste water. Particles can be coated with aluminum oxide or some other protective layer. The lifetime of the particles in the ground can be adjusted by the chemical composition and thickness of the layer. Particles may also have a paramagnetic core or layer, or they may be bonded with paramagnetic nanoparticles so that they may be concentrated before detection. Concentration is not mandatory most cases, but may facilitate the detection.

Clinical Diagnostics

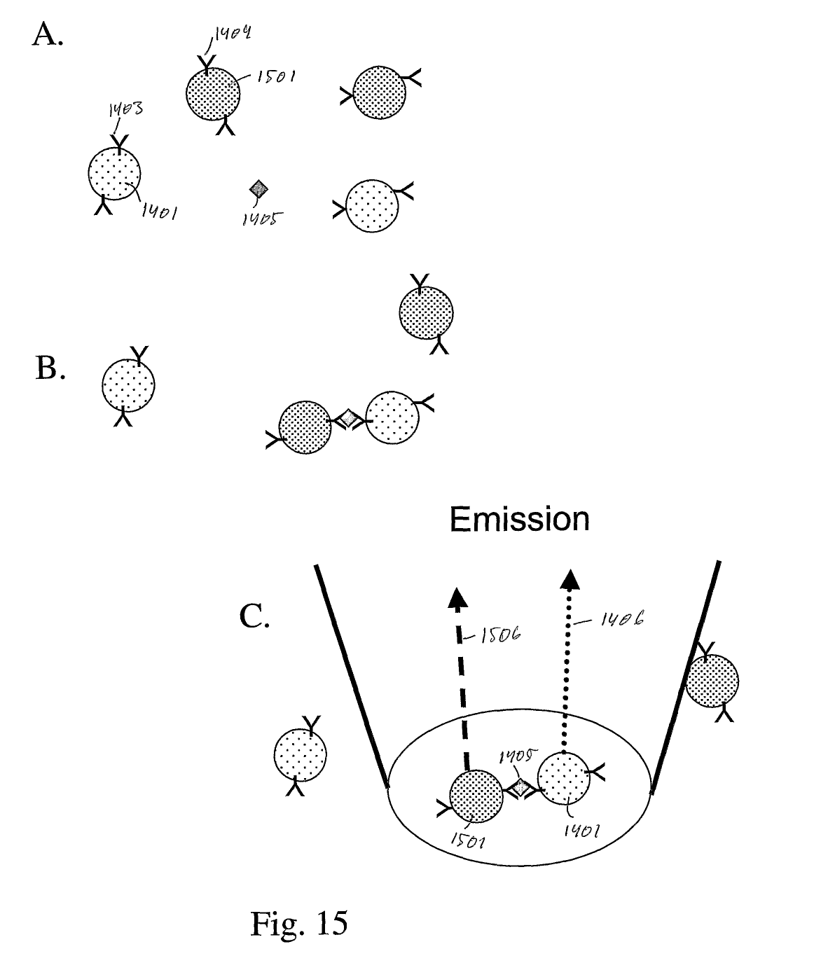

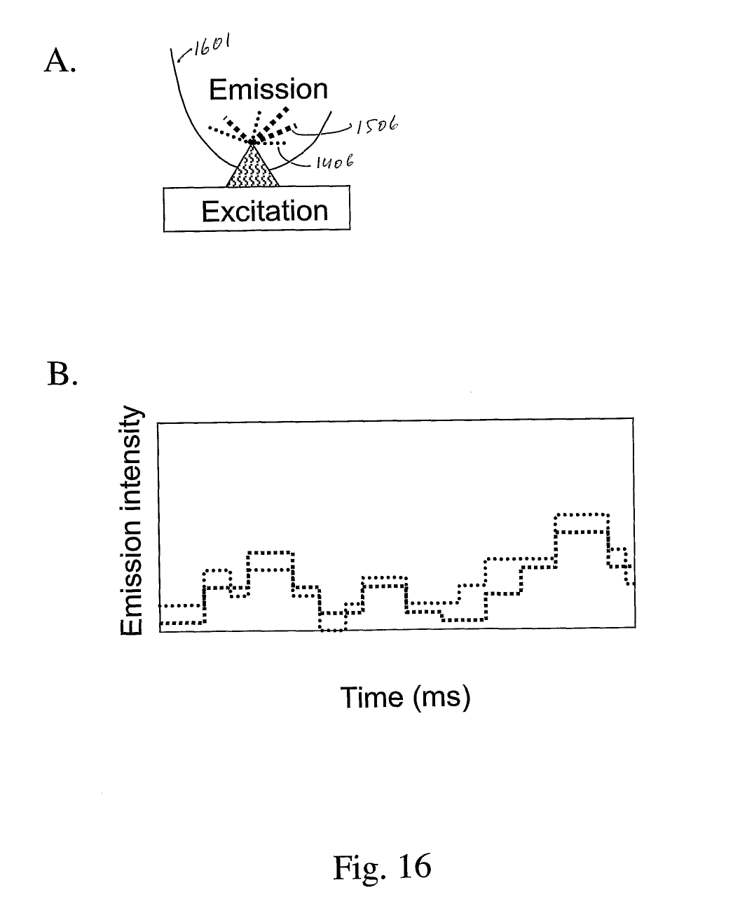

In vivo analysis of many biomolecules and pathogens will also be possible by nontoxic luminescent nanoparticles. The detection is based on dual bead formation. Dual bead method, the conjugation chemistries and other associated methods are well known in the art (Phan B C, et al., Dual bead assays including optical biodiscs and methods relating thereto. PCT Intl. Appl. 2002, priority US 2000-253283). An analyte will form a sandwich structure of two beads. The in vivo detection is best performed in tissues that are fairly transparent. One suitable site is an ear lobe (FIG. 16). Dual bead may have fluorescence energy transfer (FRET) between two particles. One is excited by an external light that can be a laser beam. If there is no FRET, the excited particle will emit light that is characteristic for that particle (FIG. 15). However, if two particles are bound, the FRET occurs, and the light is emitted from the other particle that has a longer emission wavelength. To avoid scattering and background fluorescence problems, two photon excitation may be used. Then the exciting wavelength(s) are much longer than the wavelength that is observed, and the measurement is easier.

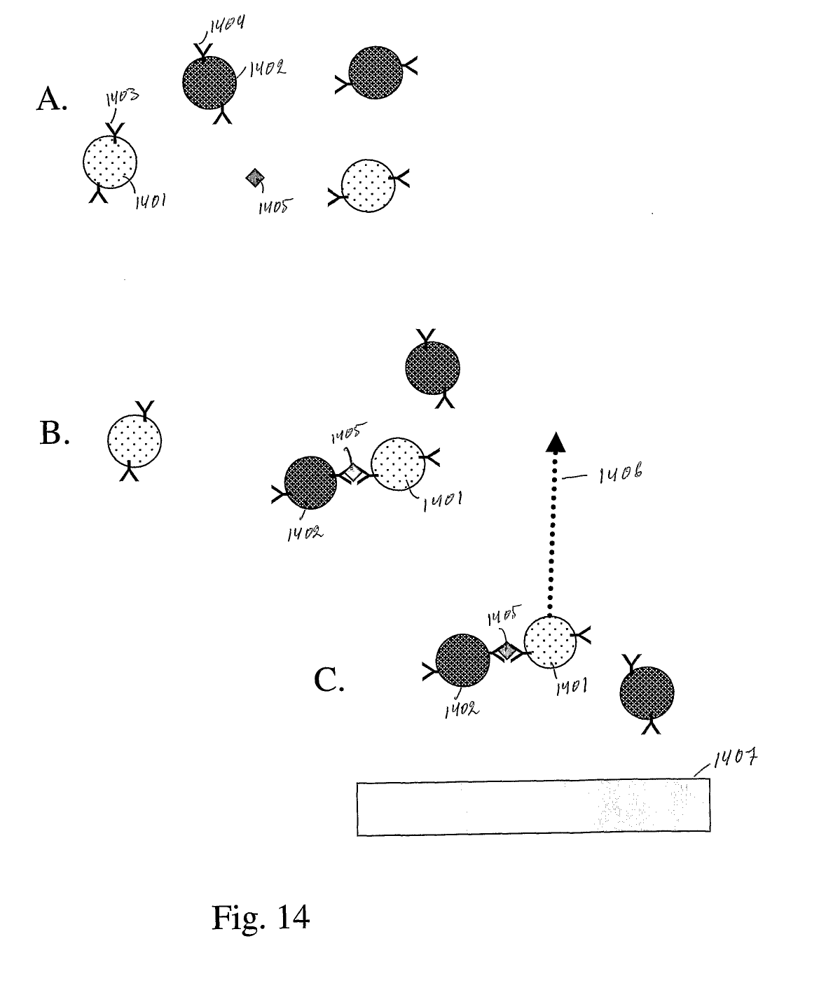

Another in vivo method is based also on the dual bead formation, but in this case one particle is luminescent 1401 and the other is paramagnetic 1402. Paramagnetic particles can be accumulated into the measurement area by a magnet 1407. The dual beads will also accumulate, and the increase of the luminescence 1406 can be measured (FIG. 14).

Gene expression and viral infection can be followed by dual beacon method of this invention. In beacons a fluorophor and quencher are attached via complementary oligonucleotide sequences to the opposite ends of the actual oligonucleotide probe so that a closed loop is formed, and the fluorescence is quenched unless the beacon binds with the target and the fluorophor and quencher are separated. In a dual beacon method two beacons must bind with the target very close with each other so that fluorescence energy transfer is possible between two fluorophors. The fluorophor that absorbs shorter wavelength light is donor and the other is acceptor. Conventional fluorophors will be bleached by light. Bleaching can be slow or fast, but it is always significant. Quantum dots will not be bleached, i.e., they maintain their emission intensity. Conventional molecular quenchers are not efficient for quantum dots, instead metallic nanoparticles, such as gold or metallic carbon nanotube fragments are efficient and are not degraded by the large amount of energy that they must receive. Thus, beacons can be constructed from a oligonucleotide probe that has a suitable complementary spacer sequence at least at one end, and has quantum dot and metallic particle attached at the opposite ends. Two of these nanoparticle beacons will form a pair can be used for ultrasensitive in vivo or in vitro DNA or RNA detection. Quantum dots must have different exitation wavelengths. Donor emission and acceptor exitation must overlap. Examples are cadmium sulfide, selenide, or telluride as donor, and lanthanide oxide particles as acceptors.

One beacon is not highly specific or sensitive, because one beacon can transiently be in an open form and give a signal. In dual beacon method both beacons must be open simultaneously, and in addition they must be in close proximity. Thus background is very small, and even a tiny signal can be detected.

Drug Delivery and Gene Therapy

Both liposomes and polymeric nanoparticles may be used for the drug delivery and genetherapy. For drug delivery purposes lipid or polymer may chosen so that it has a phase transition near or slightly above physiological temperature, preferably about 39° C. For instance, janus(6v8) forms these kinds of liposomes (Example 3). Thus, the drug will be released in the inflammation site that has a slightly elevated temperature. Biocompatible polymers are preferred for medical applications. These polymers include polyglycolic acid, polylactic acid, dextran esters, and polyaminoacid, and their block and co-polymers. Many kinds of drugs can be used. Especially lipophilic drugs, steroids, such as cortisone and some breast cancer drugs that are steroid mimics are especially advantageous.

Temperature Labels in a Cold Chain

In food transportation, storage, and sale the temperature control is often essential. It is especially important the frozen food does not thaw and freeze again. Liposomes provide a natural sensors to detect temperature changes.

In one embodiment the liposomes contain a fluorophor or quantum dot, and a quencher is outside. Above freezing point a fluorescence will be observed. Also when the liposomes are frozen for the first time, they will still show about the same fluorescence. However, the water will expand, when it is frozen, and break the liposome, at least partially. The fluorophor will leak out and the quencher will get in. As a result the fluorescence is quenched, and will stay quenched even, if the liposomes are refrozen (Example 5). These kind of liposomes can be fabricated and stored at the room temperature. When they are frozen very first time they will be activated so that, when they are thawed once the fluorescence is lost. Thus, they are ideal labels for the frozen food, biodrugs, chemicals, or other commodities that must be kept frozen. They are equally good labels for the items that can not be allowed to freeze.

There are several alternatives for the fluorophor-quencher pair, such as leuco dye-oxidizer, enzyme-substrate, and enzyme-substrate-dye. In most of these cases a color will change, when the label melts the first time.

The exact transition temperature can be adjusted by various additives. For example saturated sodium chloride solution freezes at −18° C. The saturated solutions of various salts and other chemicals are preferred, because it is easier to keep the concentrations constant, when the solutions are saturated. The liposomes may be embedded into some matrix such as polyvinyl alcohol, polymethylmethacrylate (PMMA) or like.

Printed Electronics

Nanoparticles are widely used in printed electronics. The particles produced by the methods of the present invention are also applicable in printed electronics. Especially the quantum dots, metallic nanoparticles, and nanoparticles made of conducting polymers are relevant in this connection.

### EXPERIMENTAL DETAILS

While this invention has been described in detail with reference to certain examples and illustrations of the invention, it should be appreciated that the present invention is not limited to the precise examples. Rather, in view of the present disclosure, many modifications and variations would present themselves to those skilled in the art without departing from the scope and spirit of this invention. The examples provided are set forth to aid in an understanding of the invention but are not intended to, and should not be construed to limit in any way the present invention.

### Example 1

### Fabrication of Cadmium Sulfide

First, the following solution were made: 4.44 g of sodium bis(2-ethyl hexyl)sulfosuccinate (AOT) in iso-octane in a 100 ml volumetric flask (0.1 M), 332 mg of sodium sulfide in 10 ml of water in a 10 ml volumetric flask (0.4 M), and 1146 mg of cadmium nitrate in water in a 10 ml volumetric flask (0.4 M). Two different preparations of cadmium sulfide nanoparticles were made from these stock solutions.
A. Two 10 ml aliquots of AOT solution were measured into two test tubes. Into one of these was added 80 μl of cadmium nitrate solution, and into the other 80 μl sodium sulfide solution with good mixing. The solutions were allowed to stabilize for 2 hours. After the equilibration the solutions were put into a microfluidic reactor, and using a syringe pump, the two components were pumped in 0.2 μl increments as alternating pulses via 3-way connector into a Teflon tube (2 mm diameter, 1.5 m long). The pumping speed was 0.4 ml/min for both solutions. After the solutions were pumped in, about 5 ml of pure isooctane was pumped into the tube. The receiving vial contained a 5 mM solution of 11-mercapto undecanyl amine in 10 ml of iso-propanol.
B. The second preparation was done exactly in the same way as described in A, but five times (400 μl) as much cadmium nitrate, and sodium sulfide solutions.

Both preparations were further conjugated with biotin, 10 ml of 5 mM solution in iso-propanol, using two equivalents of N-(3-dimethylaminopropyl)-N′-ethylcarbodiimide hydrochloride (EDC) and N-hydroxysuccinimide (NHS). 20 ml of water was added, iso-octane phase was separated, and the solution was dialyzed three times against 200 ml of water.

### Example 2

The fabrication was performed exactly as in Example 1, but the receiving vial did not contain any capping reagent. Instead the product solution (20 ml) as such, and 4 mg of niobium isopropoxide in 5 ml of isooctane/ethanol 4:1 were pumped into the microfluidic reactor. In each pumping cycle 0.8 μl of CdS nanoparticle solution, and 0.2 μl of niobium ethoxide solution were pumped. The product solution that contained niobium oxide coated CdS nanoparticles was put once more in the starting vial, and this time the other component was a reverse micellar solution made of adding 40 μl of poly-L-lysine (PLL, 2 mg) water solution into 10 ml of AOT base solution. In one cycle 0.5 μl of nanoparticle solution, and 0.2 μl of PLL solution were pumped in the mixing capillary. Three layer nanoparticles were isolated by adding 10 ml of water, separating the organic layer, and dialyzing the water phase three times against 200 ml of water. These CdS core nanoparticles can be attached with standard amino conjugation chemistries with other particles, biomolecules, or cells.

### Example 3

Janus(6v8) (FIG. 12) was dissolved into ethanol, concentration was 5 mg/ml. Merocyanine 540 was added into the solution to the final concentration of 0.5 mol % of janus(6v8). This solution was injected into ten fold excess of water in 0.1 μl portions. The liposomes were dialyzed in line first against 10% ethanol in water and then against pure water. Similarly, liposomes were prepared of all janus molecules in FIG. 12 with and without merocyanine 540. Especially janus(6v8) liposomes have sharp phase transition at 39° C. that can be detected with differential scanning calorimeter. The fluorescence of merocyanine 540 increased at the same temperature, which indicates that merocyanine 540 penetrates into the alkyl chain layer. Thus, these liposomes are ideal temperature activated drug carriers.

### Example 4

Polyethylene glycol polylactic acid copolymer was fabricated by reaction polyethylene glycol with L-lactide (molar ratio 1:20 or 1:40) in the presence of tin octanoate at 130° C. This polymer was dissolved into ethanol and a solution of cortisone in acetone was added, so that the amount of cortisone was 2% of the amount of the polymer. The solution was injected into fifty fold excess of water in 0.05 μl portions. The organic solvents were removed by dialysis against PBS buffer.

Poly(lactic co-glycolic acid) was added in molar ratios 1:0.5, 1:1, 1:2, and 1:4. In these cases the mixture was injected into a polyvinyl alcohol water solution. The sizes of the particles ranged from 60 nm to 180 nm depending of the amount of poly(lactic co-glycolic acid).

### Example 5

Into the merocyanine 540 containing liposomes of example 3 was added 1% of ferric chloride. The fluorescence did not change indicating that merocyanine 540 stayed inside and ferric chloride outside. The liposomes were frozen and allowed to thaw. The fluorescence had completely disappeared. This proves that the membrane is disrupted, when the sample is frozen. During thawing the contents will be released, and also the ferric chloride has access to the inside of the liposomes.

1. A method for the fabrication of nanoparticles known for a microprocessor controlled programmed pulsating addition of reagents or compounds into a microfluidic system, control of the physical conditions within the said microfluidic system, and continuous real time monitoring at least one physical property of the said nanoparticles so that the said microprocessor will maintain the said physical property within preset limits by controlling the said reagent addition and the said physical conditions.

2. A method of claim 1, in which the said physical property is luminescence excitation of the said nanoparticles.

3. A method of claim 1, in which the said physical property is luminescence emission of the said nanoparticles.

4. A method of claim 1, in which the said physical property is light scattering of the said nanoparticles.

5. A method of claim 1, in which the said physical property is the light absorption of the said nanoparticles.

6. A method of claim 1, in which the said physical property is paramagnetism of the said nanoparticles.

7. A method of claim 1, in which the said physical condition is the temperature within the said microfluidic system.

8. A method of claim 1, in which the said physical condition is the ultrasonic vibration within the said microfluidic system.

9. A method of claim 1, in which the said physical condition is the pressure within the said microfluidic system.

10. A method of claim 1, in which the said programmed pulsating addition of reagents is performed in an alternating pulses.

11. A method of claim 1, in which the said programmed pulsating addition of reagents is performed in sinusoidal pulses.

12. A method of claim 1, in which the said reagents are ionic compounds in water or solvents that are soluble in water, and a capping reagent is used.

13. A method of claim 3, in which the said nanoparticles are quantum dots.

14. A method of claim 1, in which the said reagents are in reverse micelles.

15. A method of claim 1, in which at least one of the said reagents or compounds is a polymer solubilized into a solvent that is miscible with water.

16. A method of claim 1, in which at least one of the said reagents or compounds is a amphiphilic lipid solubilized into a solvent that is miscible with water.

17. A method of claim 12, in which a resorcarene is the said capping reagent.

18. A method of claim 15, in which one of the said compounds is a drug.

19. A method of claim 18, in which lactic acid is one of the monomers in the said polymer, and the said drug is cortisone.

20. A method of claim 16, in which the said amphiphilic lipid has dendritic polar head group.

21. A nanoparticle, which is fabricated with a method of claim 1.

22. A polymeric nanoparticle that is fabricated with a method of claim 18.

23. A liposome that is fabricated with a method of claim 20.

24. A device for the fabrication of nanoparticles known for a microprocessor for programmed addition of reagents, and a microfluidic system for the formation of the said nanoparticles under controlled of physical conditions within the said microfluidic system, and a detector for a continuous real time monitoring at least one physical property of the said nanoparticles so that the said microprocessor will maintain the said physical property within preset limits by controlling the said reagent addition and the said physical conditions.

25. A device of claim 24, in which the said detector is an optical inspection unit.

26. A device of claim 24, in which the said optical inspection unit is a spectrofluorometer.

27. A device of claim 24, in which the said detector is a magnetometer.

28. A device of claim 24, in which the said programmed addition is performed with electric motor driven syringes.

29. A device of claim 24, in which the said programmed addition is performed with piezo crystal pumps.

30. A device of claim 24, in which the said programmed addition is performed with solenoid pumps.

31. A device of claim 24, in which the said control of physical conditions involves the temperature control.

32. A device of claim 24, in which the said control of physical conditions involves the control of ultrasonic vibration.

33. A device of claim 24, in which the said control of physical conditions involves the dialysis.

34. A liposome of claim 16, which contains a dye.

35. The use of the liposome of claim 34 to detect the continuity of a cold chain.

36. The use of the nanoparticle of claim 21 in in vivo diagnostics.
